(19) United States
(12) Patent Application Publication
(10) Pub. No.: US 2009/0021740 A1
(43) Pub. Date: Jan. 22, 2009

(54) OPTICAL SENSOR AND METHOD FOR MEASURING BLOOD GAS

(75) Inventors: Shen-Kan Hsiung, Tao-Yuan (TW); Jung-Chuan Chou, Tao-Yuan (TW); Tai-Ping Sun, Tao-Yuan (TW); Chih-Wei Lai, Tao-Yuan (TW); Nien-Hsuan Chou, Tao-Yuan (TW)

Correspondence Address: WPAT, PC, 7225 BEVERLY ST., ANNANDALE, VA, 22003, US

(73) Assignee: CHUNG YUAN CHRISTIAN UNIVERSITY, Tao-Yuan (TW)

(21) Appl. No.: 11/778,672

(22) Filed: Jul. 17, 2007

(51) Int. Cl. G01N 33/49; G01N 21/77

(52) U.S. Cl. 356/434; 356/39

(57) ABSTRACT

The present invention discloses an optical sensor for measuring blood gas, used to measure gas concentration in a liquid, comprising: an indicator solution; a first conduit for filling with the indicator solution wherein the first conduit passes through the liquid and the gas in the liquid can diffuse into the first conduit to react with the indicator solution so as to change the color of the indicator solution; a second conduit for receiving the reacted indicator solution in the first conduit; and a light emitter and a photodetector provided in opposition to each other at the two sides of the second conduit wherein the light emitter emits light towards the second conduit and the photodetector receives the light passing through the second conduit and the reacted indicator solution and thereby outputs a sensing signal depending on the received light intensity.

### BACKGROUND OF THE INVENTION

1. Field of the Invention

The present invention is generally related to an optical sensor and a method for measuring blood gas, and more particularly to an optical sensor and a method for measuring blood gas based on the light absorption sensing technique.

2. Description of the Prior Art

While measuring blood gas, a stable and easily continuously operational sensor is needed to measure the gas concentration in blood. However, a conventional electrode for measuring blood gas has high accuracy in measurement but is very expensive. In addition, it consumes the gas content in blood while taking measurement and has large volume so that it is not suitable for invading-type continuous measurement for human beings. Furthermore, a general biosensor is also too bulky to be used in home health care.

In addition, a conventional optical sensor for measuring blood gas has high production cost and is required to be in accord with the complicate circuit design to prevent measurement fault due to the environmental cause while taking measurement.

### SUMMARY OF THE INVENTION

The present invention provides an optical sensor for measuring blood gas, used to measure gas concentration in a liquid, comprising: an indicator solution; a first conduit for filling with the indicator solution wherein the first conduit passes through the liquid and the gas in the liquid can diffuse into the first conduit to react with the indicator solution so as to change the color of the indicator solution; a second conduit for receiving the reacted indicator solution in the first conduit; and a light emitter and a photodetector provided in opposition to each other at the two sides of the second conduit wherein the light emitter emits light towards the second conduit and the photodetector receives the light passing through the second conduit and the reacted indicator solution and thereby outputs a sensing signal depending on the received light intensity; wherein the light absorbability of the color of said indicator solution with respect to the light emitted by said light emitter decreases as the to-be-measured gas concentration in the liquid increases.

The present invention further provides an optical sensor for measuring blood gas, used to measure gas concentration in a liquid, comprising: a first container for storing the liquid; a conduit passing through the first container for filling with an indicator solution wherein the gas in the liquid can diffuse into the conduit by liquid pressure to react with the indicator solution so as to change the color of the indicator solution and the light absorbability of the reacted indicator solution decreases as the concentration of the gas diffusing in the conduit increases; a second container connected to the conduit for receiving the reacted indicator solution in the conduit; and a light emitter and a photodetector provided in opposition to each other at the two sides of the second container wherein the light emitter emits light towards the second container and the photodetector receives the light passing through the second container and the reacted indicator solution and thereby outputs a sensing signal depending on the received light intensity.

The present invention further provides a method for measuring blood gas, used to measure gas concentration in a liquid, comprising: filling an indicator solution into a conduit wherein said conduit passes through the liquid; allowing the gas in the liquid diffusing into said conduit to react with said indicator solution so as to become a reacted indicator solution wherein the light absorbability of said reacted indicator solution decreases as the concentration of the gas diffusing into said conduit increases; using a light emitter for emitting light to let the light pass through said reacted indicator solution; and using a photodetector for receiving the light passing through said reacted indicator solution and thereby outputting a sensing signal depending on the received light intensity.

The present invention further provides an optical sensor for measuring blood gas, used to measure gas concentration in a liquid, comprising: a container with an opening for storing an indicator solution; a silica gel layer for sealing said opening; and a light emitter and a photodetector provided in opposition to each other at the two sides of said container wherein said photodetector receives the light emitted from said light emitter passing through said container and said indicator solution and thereby outputs a sensing signal depending on the received light intensity.

### BRIEF DESCRIPTION OF THE DRAWINGS

FIG. 1 is a system block diagram in a preferred embodiment according to the present invention;

FIG. 2 is a schematic structure diagram of a preferred first container in FIG. 1;

FIG. 3 is a schematic diagram of a preferred sensing structure;

FIG. 4 is a schematic diagram of another preferred sensing structure; and

FIG. 5 is a flow chart illustrating a preferred measurement method according to the present invention.

### DESCRIPTION OF THE PREFERRED EMBODIMENTS

Some preferred embodiments of the present invention will now be described in greater detail in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims. In order to have clear description of the invention and better understanding for those who are skilled in the art, some part of the figures is not drawn in proportion, in which the size of some part has been exaggerated. Besides, some of irrelevant part is not shown for simplicity.

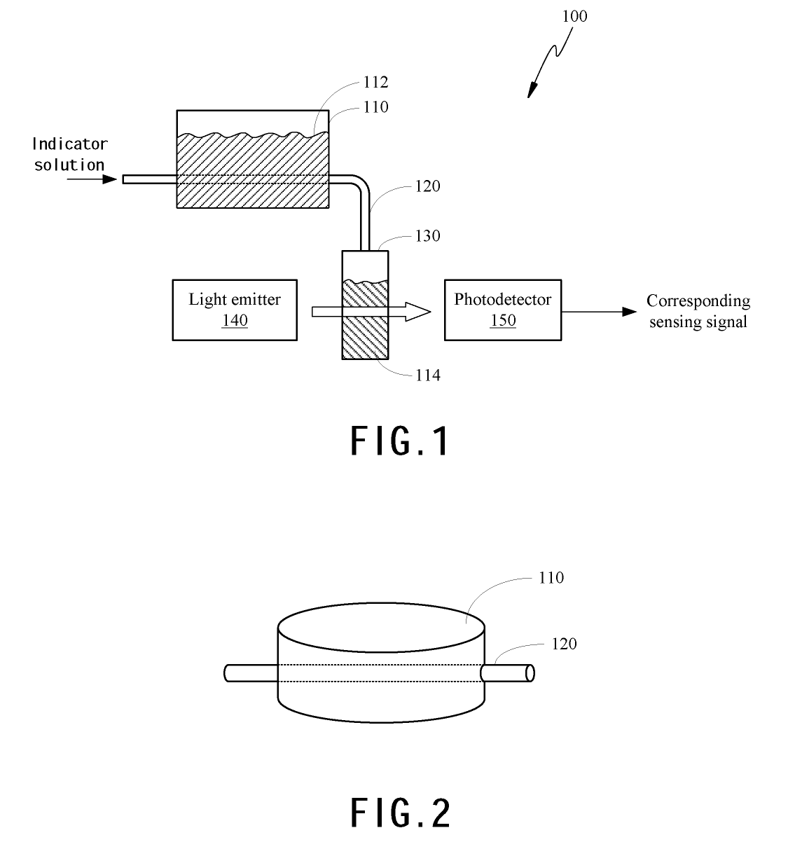

FIG. 1 is a system block diagram in a preferred embodiment 100 according to the present invention. A first container 110 (or called “sampling bottle”) stores a liquid 112. The liquid 112 is the to-be-measured liquid, such as human blood or other liquid, in this embodiment for measuring gas concentration therein. A conduit 120, for filling with an indicator solution, passes through the liquid 112. In the embodiment, the conduit 120 is of silica gel material and has tube thickness of 0.2 mm so that the gas in the liquid 112 can diffuse into the conduit 120 by liquid pressure to react with the indicator solution therein. In the embodiment, the indicator solution filled in the conduit comprises an acid-base indicator solution, such as phenol red. A preferred concentration is about 0.1313 mg/L. As the gas to be measured is gas in human being, such as carbon dioxide, the indicator solution further comprises a bicarbonate solution, such as sodium bicarbonate with concentration of 35 mM as a buffer solution to suppress acid response between carbon dioxide and the acid-base indicator and thereby to expand the measurement range of the acid-base indicator to that of human blood and to increase the measurement resolution by controlling the concentration of the buffer solution.

For further explanation of the above embodiment, when carbon dioxide in blood diffuses into the conduit 120, carbon dioxide reacts with the indicator solution (acid-base indicator). As the concentration of carbon dioxide in the measured blood is higher (That is, the amount of carbon dioxide diffusing into the conduit 120 is increased), the indicator solution changes color towards acid reaction. For example, the phenol red indicator solution changes its color towards yellow. As the concentration of carbon dioxide in the measured blood is lower (That is, the amount of carbon dioxide diffusing into the conduit 120 is decreased), the indicator solution changes color towards base reaction. For example, the phenol red indicator solution changes its color towards red. The above mentioned acid-base indicator solution and indicator solution are used only for example and not for limiting the scope of the present invention. The indicator solution changes reaction color in compliance with the gas concentration to be measured. The corresponding absorbability of the indicator solution is also changed at the same time as changing reaction color. For example in a phenol red indicator solution, as the color of the solution inclines towards yellow, the corresponding absorbability is lower (higher transparency). As the color inclines towards red, the corresponding absorbability is higher (lower transparency).

A second container 130 is connected to the conduit 120 for receiving the reacted indicator solution 114 in the conduit. In this embodiment, the second container is a quartz cuvette but it is not limited to this. The light absorbability of the reacted indicator solution 114 decreases as the concentration of carbon dioxide diffusing in the conduit 120 increases. A light emitter 140 and a photodetector 150 are provided in opposition to each other at the two sides of the second container 130. The photodetector 150 receives the light emitted from the light emitter 140 passing through the second container 130 and the reacted indicator solution 114. The photodetector 150 thereby outputs a sensing signal depending on the received light intensity. Therefore, as the light absorbability of the reacted indicator solution 114 is higher, the light received by the photodetector 150 decreases, accordingly. As the light absorbability of the reacted indicator solution 114 is lower, the light received by the photodetector 150 increases, accordingly. In this embodiment, the light emitter 140 is either a light emitting diode (LED) or a laser diode (LD) and the light emitted from the light emitter 140 is green light. A preferred wavelength is 555 nm. The photodetector 150 is a sensor to transform a light signal into a signal with the corresponding frequency. The light emitter 140 and the photodetector 150 can be surface mounting electronic components mounted on the two sides of the second container 130.

FIG. 2 is a schematic structure diagram of a preferred first container 110 in FIG. 1. The first container 110 (or called “sampling bottle”) has cylindrical structure and the conduit 120 passes through the first container 110. The conduit 120 can have circuitous structure (not shown in the figure) to pass through the first container 110 so as to increase the surface area for gas to diffuse into the conduit 120. The materials and functionalities of the first container 110 and the conduit 120 in FIG. 2 are the same as those in FIG. 1. It should be noted that the two seams at the locations that the conduit 120 passes through the first container 110 have to be sealed, such as sealed by insulation glue, and the conduit 120 has to be avoided to contact with external environment so as to avoid liquid spill and gas dissipation in the liquid.

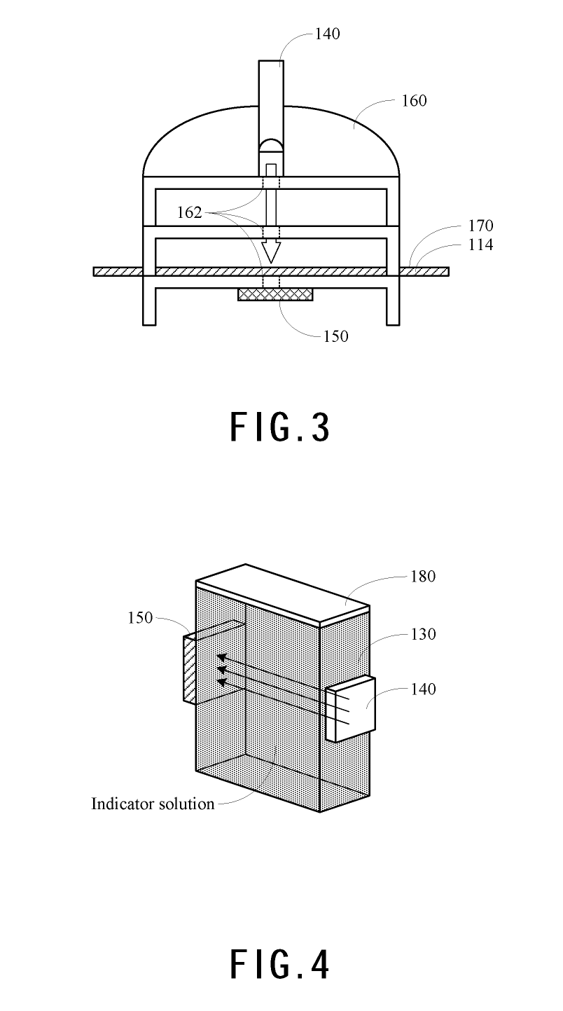

FIG. 3 is a schematic diagram of a preferred sensing structure. A portable frame 160 is used to mount the light emitter 140 and the photodetector 150. The light emitter 140 is mounted in a hole of the portable frame 160 while the photodetector 150 is mounted underneath a partition plate of the portable frame 160. The light emitter 140 is opposing to the photodetector 150 through a light passage hole 162 of a partition plate of the portable frame 160. Thus, the light emitted from the light emitter 140 passes the light passage hole 162 and then is received by the photodetector 150. A preferred diameter of the light passage hole 162 is 1 mm. A second conduit 170 is connected to the conduit 120 (also called “first conduit” in this structure) for receiving the reacted indicator solution 114 in the first conduit 120. The second conduit 170 passes through the light path between the light emitter 140 and the photodetector 150. That is, the light emitter 140 and the photodetector 150 are provided in opposition to each other at the two sides of the second conduit 170. The photodetector 150 receives the light emitted from the light emitter 140 passing through the second container 130 and the reacted indicator solution 114. The photodetector 150 thereby outputs a sensing signal depending on the received light intensity. Therefore, as the light absorbability of the reacted indicator solution 114 is higher, the light received by the photodetector 150 decreases, accordingly. As the light absorbability of the reacted indicator solution 114 is lower, the light received by the photodetector 150 increases, accordingly. In addition, in this embodiment, the second conduit 170 can be a quartz conduit to prevent from light scattering.

FIG. 4 is a schematic diagram of another preferred sensing structure. A container 130, referring to the second container 130 in FIG. 1, with an opening is used for storing an indicator solution 112 shown in FIG. 1. The opening is sealed by a silica gel layer 180. A light emitter 140 and a photodetector 150 are provided in opposition to each other at the two sides of the container 130. The photodetector 150 receives the light emitted from the light emitter 140 passing through the container 130 and the indicator solution. The photodetector 150 thereby outputs a sensing signal depending on the received light intensity. In this embodiment, the whole sensing structure is provided in the to-be-measured liquid to measure the gas concentration therein. Therefore, the gas in the liquid diffuses into the container 130 through the silica gel layer 180 to react with the indicator solution so as to change the color of the indicator solution (light absorbability). By using the light emitter 140 and the photodetector 150, the gas concentration in the liquid is then measured. In this embodiment, the container 130 is a quartz cuvette and the functionality and composition of the indicator solution are the same as that in FIG. 1. The thickness of the silica gel layer 180 is about 0.2 mm. The light emitter 140 and the photodetector 150 are surface mounting electronic components and can be a green LED and a sensor transforming a light signal into a signal with the corresponding frequency, respectively.

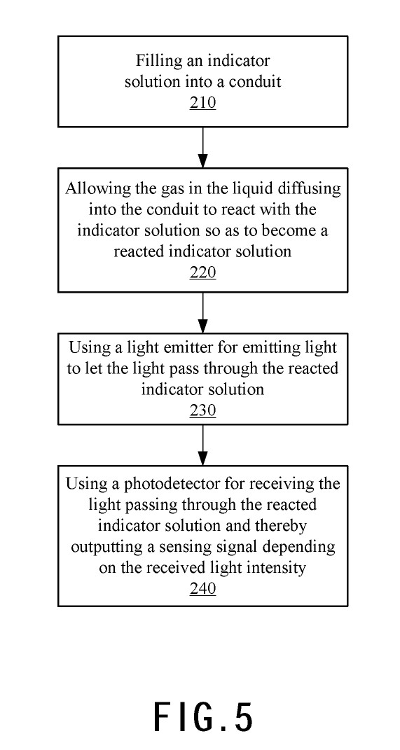

FIG. 5 is a flow chart illustrating a preferred measurement method according to the present invention. The embodiment discloses a method for measuring blood gas, used to measure gas concentration in a liquid. Step 210 is to fill an indicator solution into a conduit wherein the conduit passes through the liquid. In this embodiment, the indicator solution comprises phenol red with concentration of 0.1313 mg/L. Besides, as the liquid to be measured is human blood, the indicator solution further comprises bicarbonate with concentration of 35 mM as a buffer solution. In addition, the thickness of conduit is about 0.2 mm. Step 220 is to allow the gas in the liquid diffusing into the conduit to react with the indicator solution so as to become a reacted indicator solution wherein the light absorbability of the reacted indicator solution decreases as the concentration of the gas diffusing in the conduit increases. Step 230 is to use a light emitter for emitting light to let the light pass through the reacted indicator solution. Step 240 is to use a photodetector for receiving the light passing through the reacted indicator solution and thereby outputting a sensing signal depending on the received light intensity. In this embodiment, the light emitter can be either a green light emitting diode (LED) or laser diode (LD) with a preferred wavelength of 555 nm. The photodetector can be a sensor that transforms a light signal into a signal with the corresponding frequency. Thus, by the acid-base reaction and the light absorption technique together with optical measurement, the gas concentration in a liquid can be measured.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. An optical sensor for measuring blood gas, used to measure gas concentration in a liquid, said optical sensor comprising:
an indicator solution;
a first conduit for filling with said indicator solution wherein said first conduit passes through the liquid and the gas in the liquid can diffuse into said first conduit to react with said indicator solution so as to change the color of said indicator solution;
a second conduit for receiving said reacted indicator solution in said first conduit; and
a light emitter and a photodetector provided in opposition to each other at the two sides of said second conduit wherein said light emitter emits light towards said second conduit and said photodetector receives the light passing through said second conduit and said reacted indicator solution and thereby outputs a sensing signal depending on the received light intensity;
wherein the light absorbability of the color of said indicator solution with respect to the light emitted by said light emitter decreases as the to-be-measured gas concentration in the liquid increases.

2. The optical sensor according to claim 1, wherein said indicator solution comprises an acid-base indicator.

3. The optical sensor according to claim 2, wherein said acid-base indicator comprises phenol red.

4. The optical sensor according to claim 3, wherein the concentration of said phenol red is about 0.1313 mg/L.

5. The optical sensor according to claim 2, wherein said indicator solution further comprises bicarbonate.

6. The optical sensor according to claim 5, wherein the concentration of said bicarbonate is about 35 mM.

7. The optical sensor according to claim 1, wherein the material of said first conduit comprises silica gel.

8. The optical sensor according to claim 1, wherein the tube thickness of said first conduit is about 0.2 mm.

9. The optical sensor according to claim 1, wherein the material of said second conduit comprises quartz.

10. The optical sensor according to claim 1, wherein said light emitter comprises a light emitting diode and said light emitting diode emits green light.

11. The optical sensor according to claim 1, wherein said light emitter comprises a laser diode and said laser diode emits green light.

12. The optical sensor according to claim 1, wherein said photodetector comprises a sensor for transforming a light signal into a signal with the corresponding frequency.

13. An optical sensor for measuring blood gas, used to measure gas concentration in a liquid, comprising:
a first container for storing the liquid;
a conduit passing through said first container for filling with an indicator solution wherein the gas in the liquid can diffuse into said conduit by liquid pressure to react with said indicator solution so as to change the color of said indicator solution and the light absorbability of said reacted indicator solution decreases as the concentration of the gas diffusing in said conduit increases;
a second container connected to said conduit for receiving said reacted indicator solution in said conduit; and
a light emitter and a photodetector provided in opposition to each other at the two sides of said second container wherein said light emitter emits light towards said second container and said photodetector receives the light passing through said second container and said reacted indicator solution and thereby outputs a sensing signal depending on the received light intensity.

14. The optical sensor according to claim 13, wherein the material of said conduit comprises silica gel.

15. The optical sensor according to claim 13, wherein the tube thickness of said conduit is about 0.2 mm.

16. The optical sensor according to claim 13, wherein said indicator solution comprises an acid-base indicator.

17. The optical sensor according to claim 16, wherein said acid-base indicator comprises phenol red.

18. The optical sensor according to claim 17, wherein the concentration of said phenol red is about 0.1313 mg/L.

19. The optical sensor according to claim 16, wherein said indicator solution further comprises bicarbonate.

20. The optical sensor according to claim 19, wherein the concentration of said bicarbonate is about 35 mM.

21. The optical sensor according to claim 13, wherein said second container comprises a quartz cuvette.

22. The optical sensor according to claim 13, wherein said light emitter comprises a light emitting diode and said light emitting diode emits green light.

23. The optical sensor according to claim 13, wherein said light emitter comprises a laser diode and said laser diode emits green light.

24. The optical sensor according to claim 13, wherein said photodetector comprises a sensor for transforming a light signal into a signal with the corresponding frequency.

25. A method for measuring blood gas, used to measure gas concentration in a liquid, comprising:
filling an indicator solution into a conduit wherein said conduit passes through the liquid;
allowing the gas in the liquid diffusing into said conduit to react with said indicator solution so as to become a reacted indicator solution wherein the light absorbability of said reacted indicator solution decreases as the concentration of the gas diffusing into said conduit increases;
using a light emitter for emitting light to let the light pass through said reacted indicator solution; and
using a photodetector for receiving the light passing through said reacted indicator solution and thereby outputting a sensing signal depending on the received light intensity.

26. The method according to claim 25, wherein said indicator solution comprises phenol red with the concentration of 0.1313 mg/L.

27. The method according to claim 26, wherein said indicator solution further comprises bicarbonate with the concentration of 35 mM.

28. The method according to claim 25, wherein said conduit comprises silica gel with the tube thickness of 0.2 mm.

29. The method according to claim 25, wherein said light emitter comprises a light emitting diode and said light emitting diode emits green light.

30. The method according to claim 25, wherein said light emitter comprises a laser diode and said laser diode emits green light.

31. The method according to claim 25, wherein said photodetector comprises a sensor for transforming a light signal into a signal with the corresponding frequency.

32. An optical sensor for measuring blood gas, used to measure gas concentration in a liquid, said optical sensor, comprising:
a container with an opening for storing an indicator solution;
a silica gel layer for sealing said opening; and
a light emitter and a photodetector provided in opposition to each other at the two sides of said container wherein said photodetector receives the light emitted from said light emitter passing through said container and said indicator solution and thereby outputs a sensing signal depending on the received light intensity.

33. The optical sensor according to claim 32, wherein said container comprises a quartz cuvette.

34. The optical sensor according to claim 32, wherein said indicator solution comprises an acid-base indicator.

35. The optical sensor according to claim 34, wherein said acid-base indicator comprises phenol red.

36. The optical sensor according to claim 35, wherein the concentration of said phenol red is about 0.1313 mg/L.

37. The optical sensor according to claim 34, wherein said indicator solution further comprises bicarbonate.

38. The optical sensor according to claim 37, wherein the concentration of said bicarbonate is about 35 mM.

39. The optical sensor according to claim 32, wherein the thickness of said silica gel layer is about 0.2 mm.

40. The optical sensor according to claim 32, wherein said light emitter and said photodetector are surface mounting electronic components.

41. The optical sensor according to claim 32, wherein said light emitter comprises a light emitting diode and said light emitting diode emits green light.

42. The optical sensor according to claim 32, wherein said light emitter comprises a laser diode and said laser diode emits green light.
